(19) United States
(12) Patent Application Publication
(10) Pub. No.: US 2016/0160250 A1
(43) Pub. Date: Jun. 9, 2016

(54) HYPHOMICROBIUM SP. MICROORGANISM AND METHOD OF PRODUCING PYRROLOQUINOLINE QUINONE USING THE SAME

(71) Applicant: SUNGWUN BIO CO., LTD., Sejong (KR)

(72) Inventors: In Wha JUNG, Sejong (KR); Hyung Wook JANG, Sejong (KR); Joon Ki JUNG, Sejong (KR); In Gyu LEE, Sejong (KR); Chang Jae MAENG, Sejong (KR); Ji Hun BANG, Sejong (KR); Seog Sook NA, Sejong (KR)

(21) Appl. No.: 14/958,533

(22) Filed: Dec. 3, 2015

(30) Foreign Application Priority Data
Dec. 5, 2014 (KR) 10-2014-0173838

(51) Int. Cl. C12P 17/18; C12N 1/20

(52) U.S. Cl. CPC C12P 17/182; C12N 1/20

(57) ABSTRACT

Provided are a Hyphomicrobium sp. mutant strain SWB-P91 (KCTC12695BP) having high productivity of pyrroloquinoline quinone, and a method of producing a Hyphomicrobium sp. mutant strain SWB-P91 (KCTC12695BP), which includes inducing mutation by treating a Hyphomicrobium sp. parent strain with N-methyl-N′-nitro-N-nitroguanidine (NTG) and UV rays, and culturing the mutant strain in a medium and selecting a mutant strain with high productivity of pyrroloquinoline quinone. Also, provided is a method of mass-producing pyrroloquinoline quinone, which includes culturing a Hyphomicrobium sp. mutant strain, SWB-P91 (KCTC12695BP), adsorbing pyrroloquinoline quinone in a fermenting culture solution from the fermenting culture solution using an adsorption resin, detaching the adsorbed pyrroloquinoline quinone with an eluent; and recovering pyrroloquinoline quinone by vacuum-evaporating the detached pyrroloquinoline quinone solution.

### CROSS-REFERENCE TO RELATED APPLICATION

This application claims priority to and the benefit of Korean Patent Application No. 2014-0173838, filed on Dec. 5, 2014, the disclosure of which is incorporated herein by reference in its entirety.

### SEQUENCE STATEMENT

Incorporated by reference herein in its entirety is the Sequence Listing entitled “G15E10 D0407P US_sequence_ST25,” created Dec. 3, 2015, size of 2 kilobyte.

### BACKGROUND

1. Field of the Invention

The present invention relates to a Hyphomicrobium sp. mutant strain SWB-P91 (KCTC12695BP) having high productivity of pyrroloquinoline quinone, a method of producing the same, and a method of mass-producing pyrroloquinoline quinone.

2. Discussion of Related Art

Pyrroloquinoline quinone is a third redox coenzyme, following NAD and FAD, and the structure of pyrroloquinoline quinone was identified from a microorganism in 1979. Pyrroloquinoline quinone acts as a coenzyme having noncovalent bonds with methanol dehydrogenase of a methylotroph, alcohol dehydrogenase of an acetobacter, or glucose dehydrogenase of a Gluconobacter strain. Pyrroloquinoline quinone is present in various foods such as parsley, beans, potatoes, kiwis, papayas, etc. as well as human milk, and because problems of decrease in growth, damage to immune function, and memory failure occur when pyrroloquinoline quinone is not taken as a nutrient, it is receiving attention as a new vitamin classified as an essential nutrient (Non-Patent Document 1).

As microorganisms used to produce pyrroloquinoline quinone, Paracoccus, Protaminobacter, Pseudomonas [U.S. Pat. No. 4,994,382], Methylobacterium, Ancylobacter, Hyphomicrobium, Xanthobacter, Thiobacillus, Microcyclus or Achromobacter (Patent Document 1) may be used, and pyrroloquinoline quinone may be produced by a method of increasing an output by increasing the number of gene replication using a gene involved in the production of pyrroloquinoline quinone by Hyphomicrobium and Methylobacterium sp. microorganisms (Patent Document 2).

As an example of production of pyrroloquinoline quinone using the Hyphomicrobium sp. microorganism, in U.S. Pat. No. 5,344,768 (Patent Document 3), a method of producing pyrroloquinoline quinone at a maximum concentration of 7 mg/l from a culture supernatant obtained by culturing a Hyphomicrobium sp. microorganism in a medium containing 8 g/l of methanol for 2 days was suggested. Also, in Applied and Environmental Microbiology, 55 (5) 1209˜1213, 1989 (Non-Patent Document 2), pyrroloquinoline quinone was produced from a Hyphomicrobium sp. microorganism at a maximum concentration of 1.98 mg/l and in Applied and Environmental Microbiology, 58 (12) 3970˜3976, 1992 (Non-Patent Document 3), an example of producing pyrroloquinoline quinone at a concentration of about 900 mg/l in a 15-day culture by controlling the concentration of an iron component and the concentration of a magnesium component in the medium is shown.

A microorganism using methanol as a carbon source absorbs methanol and oxidizes it with formaldehyde. Afterward, formaldehyde is converted into serine along with glycine in cells through an anabolic metabolism of the carbon source such as a serine cycle. The first mechanism of the serine cycle, which is the conversion of formaldehyde and glycine into serine, serves as a rate-determining step of the serine cycle, and thus the growth rate is controlled. Since a lag phase is increased in the early growth of the microorganism, the lag phase stops on the second day of the growth. Also, formaldehyde produced from methanol serves as a toxic component in cells, and thus when the methanol concentration in the medium is increased, the growth is inhibited by formaldehyde produced from the methanol. Accordingly, the growth begins to be degraded at the methanol concentration of 1%, and is completely inhibited at 5%.

Meanwhile, to mass-produce pyrroloquinoline quinone within a short period through fermentation, the lag phase has to be rapidly converted into an exponential growth phase, and to this end, the rate-determining step, which is a step of forming serine in the strain, has to be actively initiated, and to maintain a growth rate of the strain, the concentration of methanol in the medium has to be maintained at a high concentration. However, when methanol is present at a predetermined concentration or higher, the growth is inhibited, and therefore there is a problem of maintaining methanol at a low concentration (0.1%). Because of this problem, it is difficult to reduce a production cost for mass-producing pyrroloquinoline quinone, and the mass production needs a long culture period.

Also, to purify pyrroloquinoline quinone, pyrroloquinoline quinone is adsorbed using a diethyl aminoethyl (DEAE)-Sephadex A-25 resin, and detached with a 1.0 M potassium chloride solution. Afterward, pyrroloquinoline quinone is precipitated for recovery from an eluent through acid precipitation. However, since the DEAE resin is expensive, a production cost is increased under the conditions of mass production, and therefore the resin is difficult to use in an individual application, and when the acid precipitation is used, a recovery time is long and a yield is decreased.

Therefore, as a result of studies to develop a process of producing pyrroloquinoline quinone using a Hyphomicrobium sp. microorganism to solve the problems described above, the inventors obtained a mutant strain having a tolerance to high concentrations of methanol, and capable of producing a large amount of pyrroloquinoline quinone within a short period even when the methanol concentration in a culture medium is maintained at a high concentration, thereby developing a method of mass-producing pyrroloquinoline quinone.

### PRIOR ART DOCUMENTS

### Patent Document

(Patent Document 1) 1. U.S. Pat. No. 4,994,382

(Patent Document 2) 2. U.S. Unexamined Patent Application Publication No. 2013/0337511

(Patent Document 3) 3. U.S. Pat. No. 5,344,768

### Non-Patent Document

(Non-Patent Document 1) Alternative Medicine Review Volume 14 (3), 268˜277, 2009)

(Non-Patent Document 2) Applied and Environmental Microbiology, 55 (5) 1209˜1213, 1989

(Non-Patent Document 3) Applied and Environmental Microbiology, 58 (12) 3970˜3976, 1992

### SUMMARY OF THE INVENTION

The present invention is directed to providing a Hyphomicrobium sp. mutant strain SWB-P91 (KCTC12695BP) having high productivity of pyrroloquinoline quinone.

Also, the present invention is directed to providing a method of producing a Hyphomicrobium sp. mutant strain SWB-P91 (KCTC12695BP) having high productivity of pyrroloquinoline quinone.

Also, the present invention is directed to providing a method of mass-producing pyrroloquinoline quinone using a Hyphomicrobium sp. mutant strain SWB-P91 (KCTC12695BP).

In one aspect, the present invention provides a Hyphomicrobium sp. mutant strain SWB-P91 (KCTC12695BP) having high productivity of pyrroloquinoline quinone.

The term “fed-batch culture” used herein refers to a culture method of continuously or intermittently providing a medium without discharging a culture solution, unlike a batch culture, and the term “batch culture” refers to a method of culturing cells in a reactor without further providing or removing nutrients after charging a medium once in an early stage.

The term “high productivity” used herein refers to higher productivity of pyrroloquinoline quinone in the mutant strain of the present invention than in a conventionally known wild-type microorganism.

In another aspect, the present invention provides a method of producing a Hyphomicrobium sp. mutant strain SWB-P91 (KCTC12695BP), which includes inducing mutation by treating a Hyphomicrobium sp. parent strain with N-methyl-N′-nitro-N-nitroguanidine (NTG) and UV rays; and selecting a mutant strain with high production of pyrroloquinoline quinone by culturing the mutant strain in a medium.

In the method of producing Hyphomicrobium sp. SWB-P91 (KCTC12695BP), first, a Hyphomicrobium sp. microorganism was cultured in a medium containing methanol after treating the microorganism with N-methyl-N′-nitro-N-nitroguanidine (NTG) and UV rays. Subsequently, grown mutant strains were cultured in a medium containing formaldehyde, and the mutant strains having the most excellent growth rates were selected. The method is used to produce a new strain by selecting the mutant strain highly producing pyrroloquinoline quinone among them in a high concentration methanol medium.

The strain growth rate of the Hyphomicrobium sp. SWB-P91 (KCTC12695BP) may be increased at high concentrations of methanol and formaldehyde.

As described above, when pyrroloquinoline quinone is produced using a Hyphomicrobium sp. mutant strain having tolerance to high concentrations of methanol, first, the stain may be cultured in a culture medium containing a high concentration of methanol, thereby maintaining the tolerance. Here, the culture medium may contain 1 to 5% methanol, and 0.06 to 30 mg/l of formaldehyde, but the present invention is not limited thereto. Also, a culture temperature may be 28 to 31° C., and a culture time may be 48 to 72 hours, but the present invention is not limited thereto. Serine may be added to the medium in order to make a lag phase of the strain shorter, and preferably, serine is added at 0.1 to 1 g/l to allow the progression to an exponential growth phase.

In still another aspect, the present invention provides a method of mass-producing pyrroloquinoline quinone, which includes: culturing a Hyphomicrobium sp. mutant strain SWB-P91 (KCTC12695BP); adsorbing pyrroloquinoline quinone in a fermenting solution using an adsorption resin from the culture fermenting solution; detaching the adsorbed pyrroloquinoline quinone using an eluent; and recovering the pyrroloquinoline quinone through vacuum evaporation of the detached pyrroloquinoline quinone solution.

A microorganism that can be used in the production of the pyrroloquinoline quinone may be a Hyphomicrobium sp. strain having tolerance to high concentration methanol and high concentration formaldehyde, and is preferably Hyphomicrobium sp. SWB-P91 (KCTC12695BP).

As described above, when pyrroloquinoline quinone is produced using a Hyphomicrobium sp. mutant strain having tolerance to high concentrations of methanol, first, the strain may be cultured in a culture medium containing a high concentration of methanol, thereby maintaining the tolerance. Here, the culture medium may contain 1 to 5% methanol, a culture temperature may be 28 to 31° C., and a culture time may be 48 to 72 hours, but the present invention is not limited thereto. Serine may be added to the medium at 0.1 to 1 g/l to allow the progression of an exponential growth phase. When the concentration of the serine is less than 0.1 g/l, the growth is decreased, and thus it is difficult to approach the exponential growth phase, and when the concentration of the serine is more than 1 g/l, a cost of the medium may be excessively increased.

Subsequently, the productivity of pyrroloquinoline quinone may be increased by controlling a concentration of methanol in a medium while the grown mutant strain is inoculated into a main fermenter, resulting in inducing fed-batch culture, and maintaining productivity of pyrroloquinoline quinone and metabolic activity of the strain until the end of fermentation. Here, the medium for producing pyrroloquinoline quinone may be provided at a suitable rate for maintaining the methanol concentration in the culture solution at 0.1 to 0.5%, and thus the culture may be continuously performed at 28 to 31° C. for 100 to 150 hours, but the present invention is not limited thereto. In the process of purifying and recovering pyrroloquinoline quinone from the fermenting solution, first, bacterial cells may be removed using a centrifugal separator, and a supernatant of the culture solution may be recovered. Afterward, a pH of the recovered culture solution may be adjusted to a pH of 1.0 to 3.5. Then, pyrroloquinoline quinone may be adsorbed by passing the culture solution through DIAION HP-20. Then, the adsorption resin to which pyrroloquinoline quinone is adsorbed may be detached with an eluent. Preferably, as the eluent, ammonia water having a concentration of 0.1 to 0.5 N may be used. Afterward, a large amount of pyrroloquinoline quinone may be recovered through vacuum evaporation. According to the above-described method, 600 mg/l of pyrroloquinoline quinone or more may be produced within 7 days.

The DIAION HP-20 resin used in the process of purifying the pyrroloquinoline quinone produced by the fermentation process is cheaper and more effectively adsorbs pyrroloquinoline quinone in a fermenting solution than a conventional DEAE-Sephadex A-25 resin. Also, a recovery rate may be improved by detaching pyrroloquinoline quinone using ammonia water, and the simple process and the decrease in production cost may be achieved through a process of removing ammonia water and recovering pyrroloquinoline quinone by vacuum-evaporating ammonia water containing the detached pyrroloquinoline quinone.

In an exemplary embodiment of the present invention, pyrroloquinoline quinone could be mass-produced through fed-batch fermentation using a Hyphomicrobium sp. microorganism grown with a high concentration of methanol. Specifically, (1) serine was added into a growth medium to reduce a lag phase and induce the progression to an exponential growth phase, and (2) a high-concentration methanol-tolerant mutant strain and (3) a mutant strain having a tolerance to high concentration formaldehyde were selected. Also, (4) the mutant strain was inoculated into a medium in a main fermenter for producing pyrroloquinoline quinone in order to grow production bacterial cells, and therefore a methanol concentration in the medium was maintained high, and the mass production of pyrroloquinoline quinone was induced. Also, (5) pyrroloquinoline quinone in the fermenting solution was adsorbed using a DIAION HP-20 adsorption resin in a process of purifying pyrroloquinoline quinone from the produced fermenting solution, and pyrroloquinoline quinone was detached from the adsorption resin by pouring ammonia water on the adsorption resin. Also, (6) a large amount of the pyrroloquinoline quinone may be recovered through vacuum evaporation of the ammonia water containing the detached pyrroloquinoline quinone.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a graph showing the comparison of degrees of growth of Hyphomicrobium sp. SWB-P91 (KCTC12695BP) strains according to the number of experiments (Y axis of the graph) in which the degree of growth of bacterial cells after culture corresponds to the range of OD values (X axis of the graph) in order to confirm the approach to an exponential growth phase in a serine-containing medium; and

FIG. 2 is a graph showing the comparison of the degrees of growth of Hyphomicrobium sp. SWB-P91 (KCTC12695BP) strains in order to confirm the approach to an exponential growth phase in a serine-free medium.

### DETAILED DESCRIPTION OF EXEMPLARY EMBODIMENTS

Hereinafter, exemplary embodiments of the present invention will be described in further detail to help in understanding the present invention. However, the following exemplary embodiments are merely provided to exemplify the present invention, not to limit the scope of the present invention. The exemplary embodiments of the present invention are provided for those of ordinary skill in the art to more fully understand the present invention.

### EXAMPLE 1

### Production of Hyphomicrobium sp. SWB-P91 (KCTC12695BP) Mutant Strain

A Hyphomicrobium sp. parent strain, Hyphomicrobium denitrificans (ATCC51888), cultured in a complete plate medium (methanol 0.2%, ammonium sulfate 0.3%, potassium monohydrogen phosphate 0.14%, disodium phosphate 0.21%, serine 0.02%, magnesium sulfate 0.1%, ferrous citrate 0.003%, calcium chloride 0.003%, manganese sulfate 0.0001%, zinc sulfate 0.002%, copper sulfate 0.00001%, agar 1.5%, pH 7.0) at 30° C. for 72 hours was inoculated into a complete liquid medium and cultured at 30° C. for 48 hours. The term “%” of the concentration of the medium means wt %. After the culture, the culture solution was centrifuged at 12000 rpm for 15 minutes, and the resultant bacterial cells were washed with saline twice. The bacterial cells were suitably diluted with the saline to have a concentration of the bacterial cells of about 1 OD (600 nm), and treated with 250 μg/ml N-methyl-N′-nitro-N-nitroguanidine (NTG) at 30° C. for 30 to 80 minutes. The treated bacterial cells were washed with saline two to three times, spread on the same medium containing 5% methanol and cultured at 30° C. for 4 to 7 days, after which a fast-grown colony was isolated. The colony was cultured in a flask medium (methanol 5%, ammonium sulfate 0.3%, potassium monohydrogen phosphate 0.14%, disodium phosphate 0.21%, serine 0.02%, magnesium sulfate 0.1%, ferrous citrate 0.003%, calcium chloride 0.003%, manganese sulfate 0.0001%, zinc sulfate 0.002%, copper sulfate 0.00001%, pH 7.0) at 30° C. for 72 hours. The mutant strain obtained thereby was spread on the agar medium containing 1 mM formaldehyde and cultured at 30° C. for 4 to 7 days, after which a fast-grown colony was isolated. The colony was cultured again in a flask medium (methanol 5%, formaldehyde 1 mM, ammonium sulfate 0.3%, potassium monohydrogen phosphate 0.14%, disodium phosphate 0.21%, serine 0.02%, magnesium sulfate 0.1%, ferrous citrate 0.003%, calcium chloride 0.003%, manganese sulfate 0.0001%, zinc sulfate 0.002%, copper sulfate 0.00001%, pH 7.0) at 30° C. for 72 hours. Among the strains cultured thereby, a strain having excellent productivity of pyrroloquinoline quinone was selected by a pyrroloquinoline quinone detection method called Hyphomicrobium SWB-P91 and deposited in the Korean Collection for Type Cultures (KCTC) of the Korean Research Institute of Bioscience & Biotechnology on Oct. 21, 2014 under the accession no. KCTC12695BP. Also, the 16S rRNA sequence (SEQ. ID. NO: 1) of Hyphomicrobium sp. SWB-P91 was identified by analyzing the base sequence of the Hyphomicrobium SWB-P91.

To detect pyrroloquinoline quinone, high performance liquid chromatography was performed using 1100 high performance liquid chromatography manufactured by Agilent, and a C18 column (4.6×250 mm, 5 μm) manufactured by Shiseido. At this time, acetonitrile-buffer 1:9 (v/v) (0.1 M KH₂PO₄ and 0.1 M HClO₄:pH 2.2 -8N NaOH) was used as a mobile phase at a flow rate of 1.0 ml/min, and the optical density was detected at 245 nm

In Tables 1 and 2, the degrees of growth between a parent strain and the mutant strain SWB-P91 of the present invention were compared by treatment with high concentrations of methanol and formaldehyde. As a result, it was confirmed that, compared to the parent strain, the mutant strain SWB-P91 of the present invention shows an increased degree of growth even at high concentrations of methanol, for example, 0.5% or higher, and high concentrations of formaldehyde, for example, 1.5 mg/l.

TABLE 1

| | |
|---|---|
| Methanol | Degree of growth |

| concentration (%) | Parent strain | SWB-P91 |
|---|---|---|
| 0.2 | +++ | +++ |
| 0.5 | ++ | +++ |
| 1 | + | ++ |
| 5 | − | − |

+++: After 72-hour culture, cells are grown at OD 3.0 or higher.
++: After 72-hour culture, cells are grown at OD 2.0 to 3.0.
+: After 72-hour culture, cells are grown at OD 1.0 to 2.0.
−: After 72-hour culture, cells are grown at OD 1.0 or less.

TABLE 2

| | |
|---|---|
| Formaldehyde | Degree of growth |

| concentration (%) | Parent strain | SWB-P91 |
|---|---|---|
| 0.06 | ++ | ++ |
| 1.5 | − | ++ |
| 6 | − | + |
| 30 | − | + |

+++: After 72-hour culture, cells are grown at OD 3.0 or higher.
++: After 72-hour culture, cells are grown at OD 2.0 to 3.0.
+: After 72-hour culture, cells are grown at OD 1.0 to 2.0.
−: After 72-hour culture, cells are grown at OD 1.0 or less.

### EXAMPLE 2

### Comparison in Productivity of Pyrroloquinoline Quinone Between Hyphomicrobium sp. SWB-P91 (KCTC12695BP) Mutant Strain and Parent Strain

1.8 l of a fermentation growth medium (methanol 1%, ammonium sulfate 0.3%, potassium monohydrogen phosphate 0.14%, disodium phosphate 0.21%, serine 0.02%, magnesium sulfate 0.1%, ferrous citrate 0.003%, calcium chloride 0.003%, manganese sulfate 0.0001%, zinc sulfate 0.002%, copper sulfate 0.00001%, pH 7.0) was poured into a 5 l small fermenter and sterilized at 121° C. for 20 minutes. 200 ml of a seed culture solution cultured in the same medium at 30° C. and 120 rpm for 48 hours was inoculated into the fermenter, and fermentation was performed under conditions of 500 rpm and 1 vvm at 30° C. for 150 hours. The pH of the fermenting solution was adjusted with ammonia water to a pH of 7, methanol was added during the fermentation in a fed-batch fermentation process, and pyrroloquinoline quinone productivity of the strain was measured. In the fed-batch culture, the methanol concentration in the culture solution was maintained at 0.5%, and at this time, the final degree of bacterial cell growth and productivity of pyrroloquinoline quinone after 150-hour fermentation were compared with those of the parent strain. Since the growth of the parent strain was inhibited at the methanol concentration of 0.2% or higher in the fed-batch culture, fermentation was carried out under the same conditions while the methanol concentration was maintained at 0.1%. As shown in Table 3, in the fermentation experiment, compared to the parent strain, the growth of the mutant strain of the present invention was maintained at the methanol concentration of 0.5% in the medium, and it was confirmed that the strain of the present invention is a more stable strain having more excellent productivity than the parent strain.

TABLE 3

| | Parent strain | SWB-P91 |
|---|---|---|
| Fermentation time (hr) | 150 | 150 |
| Amount of bacterial cells (OD 600 nm) | 52 | 43 |
| Pyrroloquinoline quinone (mg/l) | 352 | 613 |
| Productivity (mg/l/hr) | 2.3 | 4.1 |

### EXAMPLE 3

### Comparison of Growth Degrees by Serine Addition to Medium

Comparative experiments were carried out to check if stable approach from a lag phase to an exponential growth phase after the addition of serine to medium components occurred. To this end, 10% of the Hyphomicrobium SWB-P91 strain was inoculated as a seed into 30 flasks containing serine-free and 0.002% serine-containing complete liquid medium (methanol 0.2%, ammonium sulfate 0.3%, potassium monohydrogen phosphate 0.14%, disodium phosphate 0.21%, magnesium sulfate 0.1%, ferrous citrate 0.003%, calcium chloride 0.003%, manganese sulfate 0.0001%, zinc sulfate 0.002%, copper sulfate 0.00001%, pH 7.0) and cultured at 30° C. and 120 rpm for 30 hours, and then degrees of the growth in the media were compared to each other.

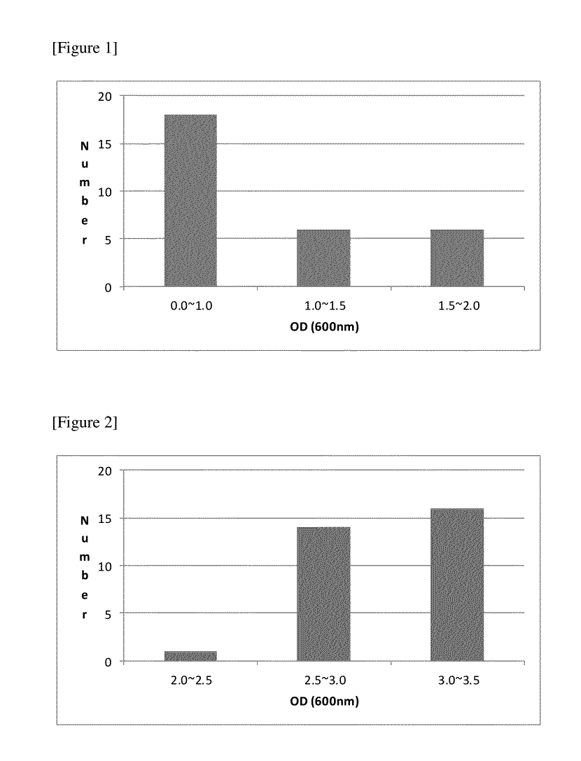

In the serine-free medium, as shown in FIG. 1, a low degree of growth and the irregular progression to the exponential growth phase were observed, but in the serine-containing medium, as shown in FIG. 2, generally stable growth and fast progression to the exponential growth phase were observed. Such results were similar to those in the experiment using the parent strain, that is, the Hyphomicrobium sp. strain. Therefore, it is concluded that the serine addition can solve the problems of instability in the early growth and the delay of the progression to the exponential growth phase, which were caused by the first step of a carbon source anabolic mechanism of a methanol-available strain, which is a serine-forming step, serving as a rate-determining step with respect to the lag phase.

### EXAMPLE 4

### Mass Purification of Pyrroloquinoline Quinone

### EXAMPLE 4-1

### Adsorption of Pyrroloquinoline Quinone on DIAION HP-20

Purification of pyrroloquinoline quinone was carried out using the fermentation culture solution prepared in Example 2. Bacterial cells were removed from 0.5 l of the cell culture using a centrifugal separator, and a supernatant was adjusted with 5 N HCl to have a pH of 1.8. The resultant product was poured into a column filled with 100 ml of a DIAION HP-20 resin to induce adsorption of pyrroloquinoline quinone. The adsorption-completed column was washed with distilled water adjusted to have a pH of 1.5 at an amount three times the amount of the resin, and detachment was carried out using 0.2 N ammonia water. As the ammonia water passed through the resin, a red band of pyrroloquinoline quinone moved along the detachment solution, and a part in which the red part was eluted was taken as a sample. Subsequently, additional detachment was carried out using 1 N ammonia water, but further elution of pyrroloquinoline quinone did not occur. Also, adsorption was carried out by pouring 0.5 l of a culture supernatant (pH 7.0) in a column filled with 100 ml of a DEAE-sepharose resin. Subsequently, the column was washed with a 0.2 M sodium chloride solution at an amount three times the amount of the resin, and detachment was carried out with a 0.65 M sodium chloride solution. A part in which the red band of pyrroloquinoline quinone was eluted was taken as a sample. Subsequently, re-elution was carried out with a 1 M sodium chloride solution, but further elution of pyrroloquinoline quinone did not occur. The comparative results of purifying pyrroloquinoline quinone using the DEAE-sepharose resin are listed in Table 4.

TABLE 4

| | DEAE-sepharose | DIAION |
|---|---|---|
| | (100 ml) | HP-20 (100 ml) |
| Adsorbed amount (mg) | 306 | 305 |
| Eluent (ml) | 39 | 68 |
| Detached amount (mg) | 197 | 232 |
| Recovery rate (%) | 64.4 | 76.0 |

### EXAMPLE 4-2

### Recovery of Pyrroloquinoline Quinone by Vacuum Evaporation

The pyrroloquinoline quinone adsorbed using the DIAION-HP-20 resin and the pyrroloquinoline quinone solution eluted with 0.2 N ammonia water according to Example 4-1 underwent vacuum evaporation. A total amount of the ammonia water was able to be removed, and about 210 mg of pyrroloquinoline quinone was able to be recovered through the vacuum evaporation. The recovery rate was 68.9%.

Meanwhile, to recover pyrroloquinoline quinone from a pyrroloquinoline quinone solution eluted using a conventional DEAE-sepharose resin, the pH was adjusted to a pH of 2.5 using a strong acid, and then acid precipitation was carried out. The pyrroloquinoline quinone obtained thereby was 149 mg, and the recovery rate was 48.6%. The comparative results are listed in Table 5.

TABLE 5

| | Acid precipitation | Vacuum evaporation |
|---|---|---|
| Recovery amount (mg) | 149 | 210 |
| Total recovery rate (%) | 48.6 | 68.9 |

Hyphomicrobium sp. SWB-P91 (KCTC12695BP) of the present invention is a new strain having effects of increasing a growth rate of bacterial cells with a high concentration of methanol and producing a large amount of pyrroloquinoline quinone within a short period. Also, a method of producing pyrroloquinoline quinone using the mutant strain is economical, and enables the process to be simplified and a large amount of pyrroloquinoline quinone to be purified.

It will be apparent to those skilled in the art that various modifications can be made to the above-described exemplary embodiments of the present invention without departing from the spirit or scope of the invention. Thus, it is intended that the present invention covers all such modifications provided they come within the scope of the appended claims and their equivalents.

[Accession No.]

Name of Deposition Organization: Korea Research Institute of Bioscience & Biotechnology

Accession No.: KCTC12695BP

Date of Deposition: 2014 Oct. 21

What is claimed is:

1. A Hyphomicrobium sp. mutant strain, SWB-P91 (KCTC12695BP), which has high productivity of pyrroloquinoline quinone.

2. A method of producing a Hyphomicrobium sp. mutant strain, SWB-P91 (KCTC12695BP), comprising:
inducing mutation by treating a Hyphomicrobium sp. parent strain with N-methyl-N′-nitro-N-nitroguanidine (NTG) and UV rays; and
culturing the mutant strain in a medium and selecting a mutant strain with high productivity of pyrroloquinoline quinone.

3. The method of claim 2, wherein the medium contains serine.

4. The method of claim 2, wherein the medium contains 0.5 to 5 wt % methanol and 0.06 to 30 mg/l of formaldehyde.

5. A method of mass-producing pyrroloquinoline quinone, comprising:
culturing a Hyphomicrobium sp. mutant strain, SWB-P91 (KCTC12695BP);
adsorbing pyrroloquinoline quinone in a fermenting culture solution from the fermenting culture solution using an adsorption resin;
detaching the adsorbed pyrroloquinoline quinone with an eluent; and
recovering pyrroloquinoline quinone by vacuum-evaporating the detached pyrroloquinoline quinone solution.

6. The method of claim 5, wherein the culture is a fed-batch culture.

7. The method of claim 6, wherein the fed-batch culture is performed to maintain a methanol concentration of the culture solution at 0.1 to 0.5 wt %.

8. The method of claim 5, wherein the eluent is ammonia water.

9. The method of claim 5, wherein the adsorption resin is DIAION HP-20.


SEQUENCE LISTING (1 sequence)

SEQ ID NO: 1 - DNA, 1125 bp, artificial sequence
  Hyphomicrobium sp. SWB-P91 16S rRNA sequence
nnnnagggggcggtctacaatgcagtcgaacgccccgcaaggggagtggcagacgggtgagtaacacgtgggaaccttccctatagtacggaatagcccagggaaacttggagtaataccgtatacgcccgaaaggggaaagaatttcgctataggatgggcccgcgtaggattagctagttggtgaggtaatggctcaccaaggcgacgatccttagctggtttgagagaacgaccagccacactgggactgagacacggcccaaactcctacgggaggcagcagtggggaatattggacaatgggcgcaagcctgatccagccatgccgcgtgagtgatgaaggccttagggttgtaaagctcttttggcggggacgataatgacggtacccgcagaataagtcccggctaacttcgtgccagcagccgcggtaatacgaaggggactagcgttgttcggaatcactgggcgtaaagcgcacgtaggcggatatgccagtcaggggtgaaatcccggggctcaacctcggaactgcccttgatacagcatgtcttgagtccgatagaggtgggtggaattcctagtgtagaggtgaaattcgtagatattaggaagaacaccggtggcgaaggcggcccactggatcggtactgacgctgaggtgcgaaagcgtggggagcaaacaggattagataccctggtagtccacgccgtagacgatggatgctagccgtcggatagcttgctattcggtggcgcagctaacgcattaagcatcccgcctggggagtacggccgcaaggttaaaactcaaaggaattgacgggggcccgcacaagcggtggagcatgtggtttaattcgacgcaacgcgaagaaccttaccagctcttgacattcactgattgccggtagagatgccggagttccagcaatggacagtgggacaggtgctgcatggctgtcgtcagctcgtgtcgtgagatgttgggttaagtcccgcaacgagcgcaaccctcgcattagttgccatcattcagttgggcactctagtgggactgccgtgatagcgggaggaaggtggggatgacgtcagtcatcatggcccttacgggctgggcta